SEQUENCE LISTING (23 sequences)

SEQ ID NO: 1 - protein, 12 aa, Unknown
  Description of Unknown Guanylin family peptide
CXXCXXXXCXXC

SEQ ID NO: 2 - protein, 18 aa, Unknown
  Description of Unknown Guanylin family peptide
XXXCEXCXXXACXXCXXX

SEQ ID NO: 3 - protein, 18 aa, Unknown
  Description of Unknown Uroguanylin B peptide
XXXCELCXNXACTGCXXX

SEQ ID NO: 4 - protein, 18 aa, Unknown
  Description of Unknown Guanylin B family peptide
XXTCEICAXAACXGCXXX

SEQ ID NO: 5 - protein, 16 aa, Homo sapiens
NDDCELCVNVACTGCL

SEQ ID NO: 6 - protein, 15 aa, Homo sapiens
PGTCEICAYAACTGC

SEQ ID NO: 7 - protein, 16 aa, Homo sapiens
NDECELCVNVACTGCL

SEQ ID NO: 8 - protein, 16 aa, Unknown
  Description of Unknown Guanylin family peptide
NDDCELCVNVACTGCL

SEQ ID NO: 9 - protein, 16 aa, Unknown
  Description of Unknown Guanylin family peptide
PGTCEICAYAACTGCL

SEQ ID NO: 10 - protein, 18 aa, Unknown
  Description of Unknown Guanylin family peptide
NDDCELCVNVACTGCLKK

SEQ ID NO: 11 - protein, 16 aa, Unknown
  Description of Unknown Guanylin family peptide
ADDCELCVNVACTGCL

SEQ ID NO: 12 - protein, 16 aa, Unknown
  Description of Unknown Guanylin family peptide
NDDCELCANVACTGCL

SEQ ID NO: 13 - protein, 16 aa, Unknown
  Description of Unknown Guanylin family peptide
NDDCELCVNAACTGCL

SEQ ID NO: 14 - protein, 16 aa, Unknown
  Description of Unknown Guanylin family peptide
NDDCELCVNVACAGCL

SEQ ID NO: 15 - protein, 16 aa, Unknown
  Description of Unknown Guanylin family peptide
NDDCELCVNVACTACL

SEQ ID NO: 16 - protein, 16 aa, Unknown
  Description of Unknown Guanylin family peptide
NDDCELCAYAACTGCL

SEQ ID NO: 17 - protein, 16 aa, Unknown
  Description of Unknown Guanylin family peptide
NDDCELCVNPACTGCL

SEQ ID NO: 18 - protein, 4 aa, Artificial Sequence
  Description of Artificial Sequence Synthetic peptide
SGSG

SEQ ID NO: 19 - protein, 18 aa, Unknown
  Description of Unknown Guanylin family peptide
XXXCEXCXXXACXGCXXX

SEQ ID NO: 20 - protein, 18 aa, Unknown
  Description of Unknown Guanylin family peptide
XXTCEICAXAACXGCXXX

SEQ ID NO: 21 - protein, 18 aa, Unknown
  Description of Unknown Guanylin family peptide
XXTCEICAXAACXGCXXX

SEQ ID NO: 22 - protein, 18 aa, Unknown
  Description of Unknown Guanylin family peptide
XXXCELCXNXACTGCXXX

SEQ ID NO: 23 - protein, 18 aa, Unknown
  Description of Unknown Guanylin family peptide
XXXCELCXNXACTGCXXX

(19) United States
(12) Patent Application Publication
(10) Pub. No.: US 2012/0028897 A1
(43) Pub. Date: Feb. 2, 2012

(54) METHODS AND COMPOSITIONS FOR THE TREATMENT OF FLUID RETENTION DISORDERS

(76) Inventors: Mark G. Currie, Sterling, MA (US); Michael F. Goy, Chapel Hill, NC (US); Marco M. Kessler, Danvers, MA (US); Nicholas G. Moss, Carrboro, NC (US); Robert M. Solinga, Cambridge, MA (US); Daniel P. Zimmer, Somerville, MA (US)

(21) Appl. No.: 13/132,262

(22) PCT Filed: Dec. 1, 2009
(86) PCT No.: PCT/US09/66227

§ 371 Date: Aug. 22, 2011

Related U.S. Application Data
(60) Provisional application No. 61/119,262, filed on Dec. 2, 2008.

(51) Int. Cl. A61K 38/10; A61P 13/12; A61P 3/00; A61P 9/00; A61P 9/12; A61P 9/04; A61P 7/10; A61P 1/16

(52) U.S. Cl. 514/9.7

(57) ABSTRACT

Methods of treating fluid retention disorders with the B isomers of guanylin family peptides are described herein. UgnB, when compared to the A isomer (UgnA), exhibits a conventional sigmoidal dose-response relationship in its natriuetic activity. Further, unlike UgnA, UgnB only weakly activates the GC-C receptor. Compositions comprising purified, or mixtures containing B isomers of guanylin family peptides are described herein are therefore useful for the treatment of fluid retention disorders.

### BACKGROUND

Uroguanylin (Ugn) and guanylin (Gn) are closely related peptides that are produced by intestinal enterochromaffin cells and goblet cells, respectively. Both peptides bind to the guanylate cyclase C (GC-C) receptor, a key regulator of fluid and electrolyte balance in the intestine. When stimulated, GC-C, which is located on the apical membrane of the intestinal epithelial surface, causes an increase in intestinal epithelial cyclic GMP (cGMP). This increase in cGMP stimulates chloride and bicarbonate efflux through the CFTR chloride channel and inhibits sodium reabsorption by a sodium hydrogen cation exchanger (NHE). GC-C is also activated by bacterially-produced heat stable toxins such as STa, STa(h), and STa(p), which are the causal agents of one form of secretory diarrhea.

In addition to their intestinal mediated responses, Ugn and Gn also circulate in plasma and elicit natriuretic responses from the kidneys. Both peptides have been proposed as volume regulatory factors that buffer acute increases in dietary salt intake by delaying sodium absorption from the intestine and increasing sodium excretion by the kidneys.

Ugn and Gn each exist as two conformationally distinct stereoisomers, termed either UgnA and UgnB or GnA and GnB. For both Ugn and Gn, the carboxy terminus appears to regulate the rate of interconversion between the A and B isomers. The rat, mouse, and opossum Ugn stereoisomers interconvert spontaneously at a rate of 1-2 cycles per sec at 37° C. By contrast, the human Ugn (huUgn) stereoisomers each have a half life of approximately 2 days at 37° C. The increased stability of human Ugn isoforms correlates with an additional C-terminal leucine residue that sterically hinders the transition between the A and B conformations. Because of this relative stability, huUgnA and huUgnB can be separated by HPLC and tested independently for activity. In such studies, huUgnA elicits robust cGMP responses when applied to cultured GC-C-expressing cells, with an EC₅₀ on the order of 10⁻⁷ M, while huUgnB is more than 100-fold less potent. Both forms of Ugn have been identified in human plasma and urine, but, given UgnB's apparent lack of biological activity, the potential physiological significance of this topoisomer has long been disregarded.

### SUMMARY OF THE INVENTION

In one aspect, the invention features a pharmaceutical composition comprising the B isomer of a guanylin family peptide. In one embodiment, the guanylin family peptide is the B isomer of a uroguanylin (Ugn) peptide or a guanylin (Gn) peptide. In another embodiment, the guanylin family peptide is UgnB or GnB. In another embodiment, the guanylin family peptide is UgnB (e.g., huUgnB) at a ratio of UgnB:UgnA of between 55:45 and 100:0 (e.g., a ratio of UgnB:UgnA of 55:45, 60:40, 65:35, 70:30, 75:25, 80:20, 85:15, 90:10, 95:5, 99:1, or greater). In another embodiment, the guanylin family peptide is GnB (e.g., huGnB) at a ratio of GnB:GnA of between 55:45 and 100:0 (e.g., a ratio of GnB:GnA of 55:45, 60:40, 65:35, 70:30, 75:25, 80:20, 85:15, 90:10, 95:5, 99:1, or greater). In another embodiment, the B isomer of a guanylin family peptide is at a non-naturally occurring ratio with the A form of the peptide, with the proviso that the B isomer is not NDDCELCVNVACTGCL, PGTCEICAYAACTGCL, NDDCELCVNVACTGCLKK, ADDCELCVNVACTGCL, NDDCELCANVACTGCL, NDDCELCVNAACTGCL, NDDCELCVNVACAGCL, NDDCELCVNVACTACL, NDDCELCAYAACTGCL, or NDDCELCVNPACTGCL (SEQ ID NOs: 8-17). In one embodiment, the pharmaceutical composition is lyophilized.

The invention also features a pharmaceutical composition comprising a UgnB (e.g., huUgnB) modified to decrease the rate of conversion of UgnB to UgnA. The invention also features a pharmaceutical composition comprising a GnB (e.g., huGnB) modified to decrease the rate of conversion of GnB to GnA.

The invention features a method for treating a disorder characterized by fluid retention in a subject, by administering an effective amount of composition comprising UgnB (e.g., huUgnB) alone, or present at a non-naturally occurring ratio with UgnA (e.g., a ratio of UgnB:UgnA of 55:45, 60:40, 65:35, 70:30, 75:25, 80:20, 85:15, 90:10, 95:5, 99:1, or greater). The invention features a method for treating a disorder characterized by fluid retention in a subject by administering an effective amount of composition comprising GnB (e.g., huGnB) in the absence of GnA, or present at a non-naturally occurring ratio with GnA (e.g., a ratio of GnB:GnA of 55:45, 60:40, 65:35, 70:30, 75:25, 80:20, 85:15, 90:10, 95:5, 99:1, or greater).

If desired, UgnB can be modified to decrease the rate of conversion to UgnA. Also, if present in the composition, UgnA can be modified to prevent conversion to UgnB. UgnB can have the amino acid sequence set forth in SEQ ID NO:5. The UgnB encoded by SEQ ID NO:5 can contain amino acid substitutions, including conserved or non-naturally occurring amino-acids. The peptide can have, for example, the following sequence: Asn Asp Glu Cys Glu Leu Cys Val Asn Val Ala Cys Thr Gly Cys Leu (SEQ ID NO:7).

Similarly, if desired, GnB can be modified to decrease the rate of conversion to GnA. Also, if present in the composition, GnA can be modified to prevent conversion to GnB. GnB can have the amino acid sequence set forth in SEQ ID NO:6. The GnB encoded by SEQ ID NO:6 can contain amino acid substitutions, including conserved or non-naturally occurring amino-acids.

The peptides and pharmaceutical compositions described herein can be used to prevent or treat a fluid retention disorder, including, for example, kidney disease, heart disease, liver disease, or hypertension. The B isomer of a guanylin family peptide can be administered alone or in combination with one or more additional agents that affect salt balance, fluid balance, or both salt and fluid balance. Such agents include diuretics (e.g., carbonic anhydrase inhibitors, thiazide-like diuretics, loop or high ceiling diuretics, and potassium-sparing diuretics; specific drugs include furosemide, bumetadine, torsemide, hydrochlorothiazide, triamterine, indapamide, ethocrinic acid, spironolactone, and metolazone).

As used herein, a guanylin family peptide is a peptide having a naturally occurring or non-naturally occurring amino acid sequence with four cysteines arranged in a characteristic pattern (Cys-Xaa-Xaa-Cys-Xaa-Xaa-Xaa-Xaa-Cys-Xaa-Xaa-Cys (SEQ ID NO:1)). In one embodiment, a guanylin family peptide contains two disulfide bonds, one between the first Cys and the third Cys of SEQ ID NO:1 and one between the second Cys and the fourth Cys of SEQ ID NO:1. In one embodiment, the A form of guanylin family peptides bind to and activate guanylate cyclase-C receptor. Guanylin family peptides include, inter alia, guanylin, uroguanylin, lymphoguanylin, and renoguanylin peptides. In one embodiment, guanylin family peptides include guanylin and uroguanylin peptides. In a further embodiment, guanylin family peptides include mammalian guanylin and uroguanylin peptides. In another embodiment, guanylin family peptides comprise the sequence Xaa₁-Xaa₂-Xaa₃-Cys-Glu-Xaa₄-Cys-Xaa₅-Xaa₆-Xaa₇-Ala-Cys-Xaa₈-Xaa₉-Cys-Xaa₁₀-Xaa₁₁-Xaa₁₂ (SEQ ID NO:2); wherein Xaa₁ is Gly, Asn, Pro, Gln, Ser, Thr, Ala, Val, Leu, Ile, Met, Phe, Trp, Tyr or is absent; Xaa₂ is Asp, Glu, Gly, H is, Asn, Ser, Gln, Thr or is absent; Xaa₃ is Thr, Glu, Asp, or Ser; Xaa₄ is Ile or Leu; Xaa₅ is Val, Ile, Ala, or Leu; Xaa₆ is Asn, Tyr, Phe, or Gln; Xaa₇ is Val, Ile, Ala, Leu or Pro; Xaa₈ is Ala, Ser or Thr; Xaa₉ is Gly or Ala; Xaa₁₀ is Leu, Ile, Phe, Trp or Tyr; Xaa₁₁ is Arg, Lys, Ala, Leu, Val, Ile, Ser, Thr, Met, Phe, Trp, Tyr, Asp, Glu, Gln, Asn or is absent; and Xaa₁₂ is Arg, Lys, Ala, Leu, Val, Ile, Ser, Thr, Met, Phe, Trp, Tyr, Asp, Glu, Gln, Asn or is absent.

“Uroguanylin B” or “UgnB” means a polypeptide having the following sequence: Xaa₁-Xaa₂-Xaa₃-Cys-Glu-Leu-Cys-Xaa₅-Asn-Xaa₆-Ala-Cys-Thr-Gly-Cys-Xaa₇-Xaa₈-Xaa₉ (SEQ ID NO:3); where Xaa₁ is Gly, Asn, Gln, Thr, or is absent; Xaa₂ is Asp, Glu, or is absent; Xaa₃ is Glu or Asp; Xaa₅ is Val or Ile; Xaa₆ is Val or Ile; Xaa₇ is Leu, Phe, or Tyr; Xaa₈ is Arg, Lys, Ala, Leu, Val, Ile, Ser, Thr, Met, Phe, Trp, Tyr, Asp, Glu, Gln, Asn or is absent; and Xaa₉ is Arg, Lys, Ala, Leu, Val, Ile, Ser, Thr, Met, Phe, Trp, Tyr, Asp, Glu, Gln, Asn or is absent. The carboxy-terminal amino acid, whether it be Xaa₇, Xaa₈, or Xaa₉ can be either a D-amino acid or an L-amino acid, and is optionally amidated.

“Guanylin B” or “GnB” means a polypeptide having the following sequence: Xaa₁-Xaa₂-Thr-Cys-Glu-Ile-Cys-Ala-Xaa₂-Ala-Ala-Cys-Xaa₃-Gly-Cys-Xaa₄-Xaa₅-Xaa₆ (SEQ ID NO: 4); where Xaa₁ is Pro, Ser, or is absent; Xaa₂ is Gly, H is, Asn, Ser, or is absent; Xaa₂ is Tyr or Phe; Xaa₃ is Ala or Thr; Xaa₄ is Leu, Phe, or Tyr; Xaa₅ is Arg, Lys, Ala, Leu, Val, Ile, Ser, Thr, Met, Phe, Trp, Tyr, Asp, Glu, Gln, Asn or is absent; and Xaa₆ is Arg, Lys, Ala, Leu, Val, Ile, Ser, Thr, Met, Phe, Trp, Tyr, Asp, Glu, Gln, Asn or is absent. The carboxy-terminal amino acid, whether it be Xaa₅ or Xaa₆, can be either a D-amino acid or an L-amino acid, and is optionally amidated.

As used herein, the “A form” and the “B form” of a guanylin family peptide are as shown in FIG. 1. Specifically, the “A form” of a guanylin family peptide is the form that, when viewed with the N-terminus of the peptide extending to the rear left and the C-terminus of the peptide extending to the front right, has the central loop formed by the four amino acids between the two central cysteines above the surface defined by the four cysteines. The “B form” is the form that, when viewed with the N-terminus of the peptide extending to the rear left and the C-terminus of the peptide extending to the front right, has the central loop formed by the four amino acids between the two central cysteines below the surface defined by the four cysteines.

“Human Ugn” or “huUgn” means a protein with the following sequence: Asn Asp Asp Cys Glu Leu Cys Val Asn Val Ala Cys Thr Gly Cys Leu (SEQ ID NO:5). “Human UgnA” or “huUgnA” means the A-isoform of huUgn; “Human UgnB” or “huUgnB” means the B isoform. The structures of the A and B isoforms are depicted in FIG. 1.

“Human Gn” or “huGn” means a protein with the following sequence: Pro Gly Thr Cys Glu Ile Cys Ala Tyr Ala Ala Cys Thr Gly Cys (SEQ ID NO:6). “Human GnA” or “huGnA” means the A-isoform of the huGn; “Human GnB” or “huGnB” means the B isoform.

The terms “UgnB” and “GnB” also include any conservative substitutions of any amino-acid residues in huUgnB or huGnB, respectively. A conservative amino acid substitution results in the alteration of an amino acid for a similar acting amino acid, or amino acid of like charge, polarity, or hydrophobicity. Among the naturally occurring amino acid substitutions generally considered conservative are:

| For Amino Acid | Code | Replace with any of |
|---|---|---|
| Alanine | Ala | Gly, Ser |
| Arginine | Arg | Lys, His |
| Asparagine | Asn | Asp, Glu, Gln, |
| Aspartic Acid | Asp | Asn, Glu, Gln |
| Glutamine | Gln | Asn, Glu, Asp |
| Glutamic Acid | Glu | Asp, Asn, Gln |
| Glycine | Gly | Ala |
| Histidine | His | Lys, Arg |
| Isoleucine | Ile | Val, Leu, Met |
| Leucine | Leu | Val, Ile, Met |
| Lysine | Lys | Arg, His |
| Methionine | Met | Ile, Leu, Val |
| Phenylalanine | Phe | Tyr, His, Trp |
| Proline | Pro | |
| Serine | Ser | Thr, Ala |
| Threonine | Thr | Ser, Met, Val |
| Tryptophan | Trp | Phe, Tyr |
| Tyrosine | Tyr | Phe, His |
| Valine | Val | Leu, Ile, Met |

Furthermore, the term huUgnB includes conservative substitutions with non-natural amino acids.

UgnB and GnB may also include additional N-terminal and/or C-terminal amino acids. In one embodiment, an additional 1, 2, 3, 4, 5, 6, 7 or 8, or more additional N-terminal amino acids may be included in UgnB or GnB. In another embodiment, an additional 1, 2, 3, 4, 5, 6, 7 or 8, or more additional C-terminal amino acids may be included in UgnB or GnB. In a further embodiment, an additional 1, 2, 3, 4, 5, 6, 7 or 8, or more additional N-terminal amino acids and an additional 1, 2, 3, 4, 5, 6, 7 or 8, or more additional C-terminal amino acids may be included in UgnB or GnB.

In all cases, the terms “UgnB” and “GnB” are meant to only include proteins that have stable activity of the B-isomer of human uroguanylin (“huUgnB activity”). A diagram of this isomer is set forth in FIG. 1. Such activity includes significant natriuretic activity and, when compared to human UgnA, weak GC-C activity. Natriuretic activity can be determined through measurements of renal sodium excretion as described herein. Significant natriuretic activity is characterized by induction of statistically significant sodium excretion at doses of UgnB or GnB above, for example, 1 nmol, 3 nmol, 5 nmol, 7 nmol, 9 nmol, 15 nmol, or greater. GC-C activity can be determined by measuring cyclic GMP synthesis in the GC-C-expressing T84 cell line as described herein. Weak GC-C activity is characterized as UgnB or GnB having 20%, 10%, 5%, 1%, 0.5%, or less activity in the GC-C activity assay as described herein compared to an equivalent amount of UgnA or GnA. Stable activity is characterized by less than 50% interconversion between A and B isomers while in solution under physiologic conditions over a period of at least 4, 5, 6, 8, 12, 24 hours, 2 days, 4 days, one week, or more.

“Modified to decrease the rate of conversion” means that, when compared to the wild-type form of the particular Gn or Ugn sequence, Gn or Ugn is modified to decrease the interconversion between the A-isoform and B-isoform.

“Protein” or “polypeptide” or “peptide” means any chain of more than two natural or unnatural amino acids, regardless of post-translational modification (e.g., glycosylation or phosphorylation), constituting all or part of a naturally-occurring or non-naturally occurring polypeptide or peptide, as is described herein.

As used herein, unless otherwise noted, a natural amino acid is a natural α-amino acid having the L-configuration, such as those normally occurring in natural eukaryotic proteins. Unnatural amino acid refers to an amino acid, which normally does not occur in eukaryotic proteins, e.g., an epimer of a natural α-amino acid having the L configuration, that is to say an amino acid having the unnatural D-configuration; or a (D,L)-isomeric mixture thereof; or a homologue of such an amino acid, for example, a β-amino acid, an α,α-disubstituted amino acid, or an α-amino acid wherein the amino acid side chain has been shortened by one or two methylene groups or lengthened to up to 10 carbon atoms, such as an α-amino alkanoic acid with 5 up to and including 10 carbon atoms in a linear chain, an unsubstituted or substituted aromatic (α-aryl or α-aryl lower alkyl), for example, a substituted phenylalanine or phenylglycine.

The present invention also provides modifications of the peptides disclosed herein. Such modifications may be linear or circular, and include peptides having unnatural amino acids. Modifications also include molecules wherein a peptide disclosed herein is non-covalently or covalently modified by substitution, chemical, enzymatic, or other appropriate means with another atom or moiety including another peptide or protein. The moiety may be “foreign” to the peptide described herein in that it is an unnatural amino acid, or in that one or more natural amino acids are replaced with another natural or unnatural amino acid. Conjugates comprising a peptide or modification described herein covalently attached to another peptide or protein are also encompassed herein. Attachment of the other moiety may involve a linker or spacer, e.g., an amino acid or peptidic linker. Modifications also include peptides wherein one, some, or all potentially reactive groups, e.g., amino, carboxy, sulfhydryl, or hydroxyl groups are in a protected form.

The atom or moiety modifying a peptide described herein may serve analytical purposes, e.g., facilitate detection of the peptide, favor preparation or purification of the peptide, or improve a relevant property of the peptide. Such properties include induction of natriuretic activity or suitability for in vivo administration, for example, solubility or stability against enzymatic degradation. Modifications include a covalent or aggregative conjugate of a peptide described herein with another chemical moiety, the modification displaying essentially the same activity as the underivatized peptide, and a “peptidomimetic small molecule” which is modeled to resemble the three-dimensional structure of any of the amino acids described herein. Examples of such mimetics are retro-inverso peptides (Chorev et al., Acc. Chem. Res. 26: 266-273, 1993). The designing of mimetics to a known pharmaceutically active compound is a known approach to the design of drugs based on a “lead” compound. This may be desirable, e.g., where the “original” active compound is difficult or expensive to synthesize, or where it is unsuitable for a particular mode of administration.

Additional examples of modifications within the above general definitions include the following:

(I) Cyclic peptides or modifications including compounds with a disulfide bridge, a thioether bridge, or a lactam. Typically, cyclic modifications containing a disulfide bond will contain two or more cysteines, which may be L-cysteine or D-cysteine. In such modifications, as an alternative to cysteine, penicillamine β,β-dimethyl-cysteine) can be used. Peptides containing thioether bridges are obtainable, e.g., from starting compounds having a free cysteine residue at one end and a bromo-containing building block at the other end (e.g., bromo-acetic acid). Cyclization can be carried out on solid phase by a selective deprotection of the side chain of cysteine. A cyclic lactam may be formed, e.g., between the γ-carboxy group of glutamic acid and the ε-amino group of lysine. As an alternative to glutamic acid, it is possible to use aspartic acid. As an alternative to lysine, ornithine or diaminobutyric acid may be employed. Also, it is possible to make a lactam between the side chain of aspartic acid or glutamic acid at the C-terminus and the α-amino group of the N-terminal amino acid. This approach is extendable to n-amino acids (e.g., β-alanine). Alternatively, glutamine residues at the N-terminus or C-terminus can be tethered with an alkenedyl chain between the side chain nitrogen atoms (Phelan et al., J. Amer. Chem. Soc. 119:455-460, 1997).

(II) Peptides disclosed herein, which are modified by substitution. In one example, one or more, for example, one or two, amino acids are replaced with another natural or unnatural amino acid, e.g., with the respective D-analog, or a mimetic. For example, in a peptide containing Phe or Tyr, Phe or Tyr may be replaced with another building block, e.g., another proteinogenic amino acid, or a structurally related analogue. Particular modifications are such that the conformation in the peptide is maintained. For example, an amino acid may be replaced by a α,α-disubstituted amino acid residue (e.g., α-aminoisobutyric acid, 1-amino-cyclopropane-1-carboxylic acid, 1-amino-cyclopentane-1-carboxylic acid, 1-amino-cyclohexane-1-carboxylic acid, 4-amino piperidine-4-carboxylic acid, and 1-amino-cycloheptane-1-carboxylic acid).

(III) Peptides described herein detectably labeled with an enzyme, a fluorescent marker, a chemiluminescent marker, a metal chelate, paramagnetic particles, biotin, or the like. In such modifications, the peptide is bound to the conjugation partner directly or by way of a spacer or linker group, e.g., a (peptidic) hydrophilic spacer. Advantageously, the conjugate is attached at the N- or C-terminal amino acid. For example, biotin may be attached to the N-terminus of a peptide disclosed herein via a serine residue or the tetramer Ser-Gly-Ser-Gly.

(IV) Peptides described herein carrying one or more protecting groups at a potentially reactive side group, such as amino-protecting group, e.g., acetyl, or a carboxy-protecting group. For example, the C-terminal carboxy group of a compound of the invention may be present in form of a carboxamide function. Suitable protecting groups are commonly known in the art. Such groups may be introduced, for example, to enhance the stability of the compound against proteolytic degradation.

(V) In some embodiments one or both members of one or more pairs of Cys residues which normally form a disulfide bond can be replaced by homocysteine, penicillamine, 3-mercaptoproline (Kolodziej et al. 1996 Int J Pept Protein Res 48:274); β,β dimethylcysteine (Hunt et al. 1993 Int J Pept Protein Res 42:249) or diaminopropionic acid (Smith et al. 1978 J Med Chem 21:117) to form alternative internal cross-links at the positions of the normal disulfide bonds. In addition, one or more disulfide bonds can be replaced by alternative covalent cross-links, e.g., an amide linkage (—CH₂CH(O)NHCH₂— or —CH₂NHCH(O)CH₂—), an ester linkage, a thioester linkage, a lactam bridge, a carbamoyl linkage, a urea linkage, a thiourea linkage, a phosphonate ester linkage, an alkyl linkage (—CH₂CH₂CH₂CH₂—), an alkenyl linkage (—CH₂CH═CHCH₂—), an ether linkage (—CH₂CH₂OCH₂— or —CH₂OCH₂CH₂—), a thioether linkage (—CH₂CH₂SCH₂— or —CH₂SCH₂CH₂—), an amine linkage (—CH₂CH₂NHCH₂— or —CH₂NHCH₂CH₂—) or a thioamide linkage (—CH₂CH(S)HNHCH₂— or —CH₂NHCH(S)CH₂—). For example, Ledu et al. (Proc Nat'l Acad. Sci. 100:11263, 2003) describe methods for preparing lactam and amide cross-links. Schafmeister et al. (J. Am. Chem. Soc. 122:5891, 2000) describes stable, hydrocarbon cross-links.

Histidyl residues are generally modified by reaction with diethylprocarbonate at pH 5.5-7.0 because this agent is relatively specific for the histidyl side chain. Para-bromophenacyl bromide also is useful; the reaction may be performed in 0.1 M sodium cacodylate at pH 6.0.

Lysinyl and amino terminal residues are reacted with succinic or other carboxylic acid anhydrides. Modification with these agents has the effect of reversing the charge of the lysinyl residues. Other suitable reagents for modifying α-amino-containing residues include imidoesters such as methyl picolinimidate; pyridoxal phosphate; pyridoxal; chloroborohydride; trinitrobenzenesulfonic acid; O-methylissurea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Modification of arginine residues requires that the reaction be performed in alkaline conditions because of the high pKₐ of the guanidine functional group. Furthermore, these reagents may react with the groups of lysine as well as the arginine epsilon-amino group.

Carboxyl side groups (aspartyl or glutamyl) are selectively modified by reaction with carbodiimides (R′—N—C—N—R′) such as 1-cyclohexyl-3-(2-morpholinyl-(4-ethyl)carbodiimide or 1-ethyl-3 (4 azonia 4,4-dimethylpentyl)carbodiimide. Aspartyl and glutamyl residues can also be converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Polypeptides or modifications thereof may be fused or attached to another protein or peptide, e.g., as a glutathione-S-transferase (GST) fusion polypeptide. Other commonly employed fusion polypeptides include, but are not limited to, maltose-binding protein, Staphylococcus aureus protein A, polyhistidine, and cellulose-binding protein.

A “peptidomimetic small molecule” of a peptide means a small molecule that exhibits substantially the same UgnB or GnB activity as the peptide itself.

A “substantially pure polypeptide” is a polypeptide or peptide that has been separated from the components that naturally accompany it. Typically, the polypeptide is substantially pure when it is at least 60%, by weight, free from the proteins and naturally-occurring organic molecules with which it is naturally associated. In further embodiments, the polypeptide is UgnB or GnB polypeptide that is at least 75%, at least 80%, at least 85%, at least 90%, at least 95%, and, in yet a further embodiment, at least 99%, by weight, pure. A substantially UgnB or GnB polypeptide may be obtained, for example, by extraction from a natural source (e.g., from enterochromaffin cells) by expression of a recombinant nucleic acid encoding UgnB or GnB, or by chemically synthesizing the polypeptide. Purity can be measured by any appropriate method, e.g., by column chromatography, polyacrylamide gel electrophoresis, or HPLC analysis. Further, UgnB or GnB can be separated from the UgnA or GnA isomer, respectively, using, for example, HPLC as described herein.

A protein is substantially free of naturally associated components when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides include those derived from eukaryotic organisms that are synthesized in E. coli or other prokaryotes.

“Treating” means administering or prescribing a pharmaceutical composition for the treatment or prevention of a disorder characterized by fluid retention.

“Subject” means any animal (e.g., a human). Other animals that can be treated using the methods, compositions, and kits described herein include horses, dogs, cats, pigs, goats, rabbits, hamsters, monkeys, guinea pigs, rats, mice, lizards, snakes, sheep, cattle, fish, and birds.

### BRIEF DESCRIPTION OF THE DRAWINGS

FIG. 1 is a graph showing LC-MS analysis of a calibration sample that contained similar amounts of huUgnA and huUgnB. The dashed trace is the UV absorbance profile and the solid trace is the extracted ion chromatogram (m/z 1667-1669) from a full MS scan. In positive ion mode, the m/z ratio for huUgn is 1667.6. For both traces, the earlier-eluting peak is huUgnA and the later-eluting peak is huUgnB. The two traces are offset by ˜0.8 mM because the UV detector is slightly upstream of the MS detector. Note that both traces have essentially identical huUgnA:huUgnB peak ratios.

FIG. 1 inset is a schematic showing the conformation of huUgnA and huUgnB.

FIG. 2A is a graph showing cGMP response in T84 cells as a function of peptide concentration in the presence of huUgnA and huUgnB. Each data point represents the mean value (±standard error) for 9 experiments with huUgnA (filled symbols) and 3 experiments with huUgnB (open symbols). The curves are fit with the log(agonist) vs dose equation (see the Methods), using an EC₅₀ of 1.8×10⁻⁷ M for huUgnA and an EC₅₀ of 1.5×10⁻⁵ M for huUgnB.

FIG. 2B is a graph showing the stimulatory effect of huUgnA and huUgnB on T84 cells. The bars show (from left to right) the cyclic GMP levels in cells treated with control medium, with 25 nM huUgnA, with 180 nM huUgnB, or with a combination of the two peptides (mean±sem, n=3).

FIG. 2C is a graph showing the amount of “UgnA like” activity as a function of time in cells treated with either huUgnA and huUgnB. The A and B isomers (filled and open circles, respectively) interconvert slowly in vitro. Peptides were incubated at 50° C. in 1 mM citrate buffer (pH 4) for either 0, 24, or 48 hr. Samples were then diluted into bioassay medium, the pH was adjusted to 7.0, and activity was measured in the T84 cell bioassay. A huUgnA standard curve was generated in the same assay, and cyclic GMP responses were converted to recoveries of “UgnA-like” peptide by interpolation on this standard curve.

FIG. 3A is a graph showing urinary excretion of “UgnA like” activity as a function of time in animals treated with the either huUgnA (filled symbols) or huUgnB (open symbols). huUgn was infused into an anesthetized rat during the period indicated by the horizontal bar. Urine was obtained continuously over a series of 14 sequential 20 min clearance periods, before, during, and after the peptide infusion. A sample of the urine acquired during each clearance period was evaluated for activity in the T84 cell bioassay. The resulting cyclic GMP responses were then converted to apparent recoveries of “UgnA-like” peptide by interpolation on a huUgnA standard curve (constructed as in FIG. 1A). Appropriate dilutions were chosen to ensure that the activity in each sample would not saturate this assay.

FIG. 3A inset is a graph showing the huUgnB data replotted on an expanded Y-axis. The low levels of activity observed after huUgnB infusion most likely represent weak responses to very high levels of huUgnB. The black and white arrowheads mark time points that were chosen for LC-MS analysis.

FIG. 3B is a graph showing total urinary excretion of “UgnA like” activity in animals treated with the indicated Ugn. A number of independent experiments, identical to those illustrated in FIG. 3A, were performed for each isomer. For each experiment, the activity recovered in the urine was summed over clearance periods 3-8 to give the total recovery, and the mean total recovery for each isomer is given by the bars (filled for huUgnA and open for huUgnB, mean±sem, n=6).

FIGS. 4A and 4B are extracted ion chromatograms (m/z 1667-1669) from a full MS scan of urine that was obtained after infusion with either the A or B form of huUgn. FIG. 4A shows LC-MS analysis of a urine sample obtained from a huUgnA-infused animal during collection period 5 (marked by the black arrowhead in FIG. 3A). The LC retention time of the prominent peak (10.61 min) corresponds to that of huUgnA, while the LC retention time of the minor peak (10.95) corresponds to that of huUgnB. FIG. 4B shows LC-MS analysis of a urine sample obtained from a huUgnB-infused animal during collection period 5 (marked by the white arrowhead in FIG. 3A). The LC retention time of the prominent peak (10.99 min) corresponds to that of huUgnB, while the LC retention time of the minor peak (10.63) corresponds to that of huUgnA.

FIG. 5 is a series of graphs showing a time course of sodium excretion (UNaV, nEq/min/gKW) during infusions of huUgnA (filled squares, left column) or huUgnB (filled circles, right column) in the amounts indicated (nmol/kg BW). Peptides were infused in 0.6 ml of isotonic saline over 60 min during the time periods indicated by the horizontal bars. Rats in the control group (open triangles) received isotonic saline alone during the infusion period. Values are means±sem for each 20 min clearance period. ANOVA testing with post hoc comparisons showed a significant increase only for huUgnA 25 mol/kg in the infusion period (p<0.05) and the post infusion period (p<0.001). huUgnB generated a significant natriuretic response in the post infusion periods after 18, 35, 70, and 140 nmol/kg (p<0.001 for all).

FIG. 6A is a graph showing net cumulative sodium excretion (total peptide stimulated output minus corresponding control output) over a three hour period during and after huUgnB infusions, plotted as a function of infused dose. The peptide was infused over the first hour in amounts of 9, 18, 35, 70, and 140 nmol/kg to provide each data point. Control value is shown as 0 nmol/kg. The curve is fitted to a log agonist response curve and shows an ED₅₀ of 19 nmol/kg.

FIG. 6B is a graph showing cumulative sodium excretion for a 3 hour period during and after infusions of huUgnA (open symbols) or ST-core (filled symbols). huUgnA was infused at 12, 25, 50, 100, and 200 nmol/kg. ST-core was infused at 17, 32, 66, 133, and 266 nmol/kg. The curves were fitted by a cubic spline algorithm.

FIG. 7 is a graph showing net cumulative sodium excretion (total peptide stimulated output minus corresponding control output) over a three hour period during and after intravenous peptide infusions. Peptides were infused during the first hour in the following amounts and combinations: A25=huUgnA 25 nmol/kg; B35=huUgnB 35 nmol/kg; A100+B35=huUgnA 100 nmol/kg combined with huUgnB 35 nmol/kg; A25+B35=huUgnA 25 nmol/kg combined with huUgnB 35 nmol/kg. The natriuretic response to A25+B35 was not different from the natriuresis evoked by A25 or B35 alone. The response to A100+B35 was not different from the response to A100 but significantly less than the response to B35 alone (*p<0.05).

FIG. 8 is a series of graphs showing time courses of potassium excretion (UKV, nEq/min/gKW) during infusions of huUgnA (filled squares, left column) or huUgnB (filled circles, right column) in the amounts indicated (nmol/kg BW). Peptides were infused in 0.6 ml of isotonic saline over 60 min during the time periods indicated by the horizontal bars. Rats in the control group (open triangles) received isotonic saline alone during the infusion period. Values are means±sem for each 20 min clearance period. ANOVA testing with post hoc comparisons showed a significant increase (p<0.05) in the post infusion period only after 140 nmol/kg huUgnB.

### DETAILED DESCRIPTION OF THE INVENTION

Methods of treating fluid retention disorders with the B isomers of guanylin family peptides are described herein. UgnB, when compared to the A isomer (UgnA), exhibits a conventional sigmoidal dose-response relationship in its natriuetic activity. Further, unlike UgnA, UgnB only weakly activates the GC-C receptor. Compositions comprising purified, or mixtures containing B isomers of guanylin family peptides are described herein are therefore useful for the treatment of fluid retention disorders.

### Guanylin Family Peptides

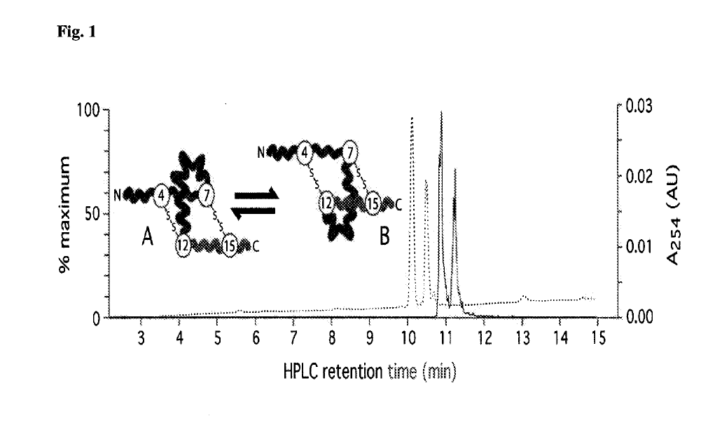

Ugn and Guanylin (Gn) are 13-16 amino acid peptides that share a distinctive ring structure produced by two disulfide bonds: one disulfide bond between the first and the third cysteines of the core guanylin family peptide motif (SEQ ID NO:1) and a second disulfide bond between the second and the fourth cysteines of the core guanylin family peptide motif. For example, in huUgn (SEQ ID NO:5), the ring structure is formed by disulfide bonds between the cysteines at positions 4 and 12 and positions 7 and 15. The central loop (formed, for example, by amino acids 8-11 in huUgn) can be positioned either above or below the surface formed by the 4 cross-linked cysteines, resulting in two conformationally distinct A and B topoisomers. The structures of these isomers are depicted in FIG. 1.

This type of isomerism is unique among mammalian peptides and, in the rat, mouse, and opossum, interconversions between the two conformations of Gn and Ugn occur at a rate of 1-2 cycles per sec at 37° C. and neutral pH. While the structure and interconversion rate of human Gn is similar to its rat counterpart, human Ugn has an additional leucine residue that extends the C terminus and sterically hinders the transition between the A and B conformations, increasing the half-life of each form to about 2 days at 37° C. Because of this relative stability, human UgnA and UgnB can be separated by HPLC and tested independently for activity. In such studies, UgnA elicits robust responses when applied to cultured GC-C-expressing cells, with an EC₅₀ on the order of 10⁻⁷ M, while UgnB is more than 100-fold less potent.

The invention features administration of the B isomer of guanylin family peptides. This guanylin family peptide can be purified human Uroguanylin B (huUgnB) or it can be huUgnB or another guanylin family peptide modified to stabilize the B-isoform.

A guanylin family peptide is a peptide having a naturally occurring or non-naturally occurring amino acid sequence with four cysteines arranged in a characteristic pattern (Cys-Xaa-Xaa-Cys-Xaa-Xaa-Xaa-Xaa-Cys-Xaa-Xaa-Cys (SEQ ID NO:1)), for example, the guanylin family peptide can contain the following sequence:
Xaa₁-Xaa₂-Xaa₃-Cys-Glu-Xaa₄-Cys-Xaa₅-Xaa₆-Xaa₇-Ala-Cys-Xaa₃-Xaa₉-Cys-Xaa₁₀-Xaa₁₁-Xaa₁₂ (SEQ ID NO:2); wherein Xaa₁ is Gly, Asn, Pro, Gln, Ser, Thr, Ala, Val, Leu, Ile, Met, Phe, Trp, Tyr or is absent; Xaa₂ is Asp, Glu, Gly, His, Asn, Ser, Gln, Thr or is absent; Xaa₃ is Thr, Glu, Asp, or Ser; Xaa₄ is Ile or Leu; Xaa₅ is Val, Ile, Ala, or Leu; Xaa₆ is Asn, Tyr, Phe, or Gln; Xaa₇ is Val, Ile, Ala, Leu or Pro; Xaa₈ is Ala, Ser or Thr; Xaa₉ is Gly or Ala; Xaa₁₀ is Leu, Ile, Phe, Trp or Tyr; Xaa₁₁ is Arg, Lys, Ala, Leu, Val, Ile, Ser, Thr, Met, Phe, Trp, Tyr, Asp, Glu, Gln, Asn or is absent; and Xaa₁₂ is Arg, Lys, Ala, Leu, Val, Ile, Ser, Thr, Met, Phe, Trp, Tyr, Asp, Glu, Gln, Asn or is absent.

The A form of guanylin family peptides can bind to and activate guanylate cyclase-C receptor (GC-C receptor). Guanylin family peptides include, guanylin (Gn), uroguanylin (Ugn), lymphoguanylin, and renoguanylin peptides.

Ugn can have the sequence:
Xaa₁-Xaa₂-Xaa₃-Cys-Glu-Leu-Cys-Xaa₅-Asn-Xaa₆-Ala-Cys-Thr-Gly-Cys-Xaa₇-Xaa₈-Xaa₉ (SEQ ID NO:3); where Xaa₁ is Gly, Asn, Gln, Thr, or is absent; Xaa₂ is Asp, Glu, or is absent; Xaa₃ is Glu or Asp; Xaa₅ is Val or Ile; Xaa₆ is Val or Ile; Xaa₇ is Leu, Phe, or Tyr; Xaa₈ is Arg, Lys, Ala, Leu, Val, Ile, Ser, Thr, Met, Phe, Trp, Tyr, Asp, Glu, Gln, Asn or is absent; and Xaa₉ is Arg, Lys, Ala, Leu, Val, Ile, Ser, Thr, Met, Phe, Trp, Tyr, Asp, Glu, Gln, Asn or is absent. The carboxy-terminal amino acid, whether it be Xaa₇, Xaa₈, or Xaa₉ can be either a D-amino acid or an L-amino acid, and is optionally amidated.

huUgn has the sequence:
(SEQ ID NO: 5)
Asn Asp Asp Cys Glu Leu Cys Val Asn Val Ala Cys
Thr Gly Cys Leu

Guanylin can have the sequence:
Xaa₁-Xaa₂-Thr-Cys-Glu-Ile-Cys-Ala-Xaa₂-Ala-Ala-Cys-Xaa₃-Gly-Cys-Xaa₄-Xaa₅-Xaa₆ (SEQ ID NO: 4); where Xaa₁ is Pro, Ser, or is absent; Xaa₂ is Gly, His, Asn, Ser, or is absent; Xaa₂ is Tyr or Phe; Xaa₃ is Ala or Thr; Xaa₄ is Leu, Phe, or Tyr; Xaa₅ is Arg, Lys, Ala, Leu, Val, Ile, Ser, Thr, Met, Phe, Trp, Tyr, Asp, Glu, Gln, Asn or is absent; and Xaa₆ is Arg, Lys, Ala, Leu, Val, Ile, Ser, Thr, Met, Phe, Trp, Tyr, Asp, Glu, Gln, Asn or is absent. The carboxy-terminal amino acid, whether it be Xaa₅ or Xaa₆, can be either a D-amino acid or an L-amino acid, and is optionally amidated.

huGn has the sequence:
(SEQ ID NO: 6)
Pro Gly Thr Cys Glu Ile Cys Ala Tyr Ala Ala Cys
Thr Gly Cys.

Further sequences of Ugn and Gn are set forth in PCT Application Publication No. WO2007/101158, which is hereby incorporated by reference in its entirety.

### Indications

The methods and compositions of the invention are useful for treating disorders that are characterized as by abnormal fluid and/or salt retention. Examples of such disorders are kidney disease or dysfunction, (including chronic glomerular nephritis and chronic renal failure), heart disease or heart failure, (including edema caused by congestive heart disease), liver disease (including cirrhosis of the liver), and hypertension. The methods and compositions of the invention are also useful for treating patients who would benefit from a diuretic drug but are not responsive to conventional diuretics.

In one embodiment, the methods and compositions of the invention are useful for treating a fluid retention disorder selected from heart failure, hypertension, salt dependent forms of high blood pressure, hepatic edema, liver cirrhosis, acute renal failure, renal insufficiency, nephrotic edema, glomerulonephritis, pyelonephritis, kidney failure, chronic renal failure, nephritis, nephrosis, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic kidney disease, medullary cystic disease, medullary sponge kidney, hereditary nephritis, and nail-patella syndrome. In a further embodiment, the fluid retention disorder is heart failure. In yet a further embodiment, the heart failure is congestive heart failure, acute heart failure or acute congestive heart failure. Yet further, the heart failure is acute decompensated congestive heart failure. In another embodiment, the fluid retention disorder is polycystic kidney disease. In a further embodiment, the polycystic kidney disease is autosomal dominant polycystic kidney disease (ADPKD) or recessive autosomal recessive polycystic kidney disease (ARPKD). In another embodiment, the methods and compositions of the invention increase natriuresis and/or diuresis.

### Formulations

The invention features administration of either substantially pure UgnB or the administration of UgnB formulated in a non-naturally occurring mixture with UgnA. For example, the ratio of UgnB to UgnA in such a mixture can be 55:45, 60:40, 65:35, 70:30, 75:25, 80:20, 85:15, 90:10, 95:5, 99:1, or greater.

The peptides of the invention can be formulated together with (or administered in conjunction with) other pharmacological agents. Such agents include, common classes of diuretics including carbonic anhydrase inhibitors, thiazide and thiazide-like diuretics, loop (or high-ceiling) diuretics, and potassium-sparing diuretics. Specific examples of such diuretics include, but are not limited to, furosemide, bumetadine, torsemide, hydrochlorothiazide, triamterine, indapamide, ethocrinic acid, spironolactone, and metolazone.

The compositions and methods described herein can be used in combination therapy with an anti-hypertensive or natriuretic agent including but not limited to:

(1) diuretics, such as thiazides, including chlorthalidone, chlorthiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; carbonic anhydrase inhibitors, osmotics (such as glycerin) and aldosterone antagonists, such as spironolactone, epirenone, and the like;

(2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like;

(3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like;

(4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like;

(5) neutral endopeptidase inhibitors such as omapatrilat, cadoxatril and ecadotril, fosidotril, sampatrilat, AVE7688, ER4030, and the like;

(6) endothelin antagonists such as tezosentan, A308165, and YM62899, and the like;

(7) vasodilators such as hydralazine, clonidine, minoxidil, and nicotinyl alcohol, and the like;

(8) angiotensin II receptor antagonists such as aprosartan, candesartan, eprosartan, irbesartan, losartan, olmesartan, pratosartan, tasosartan, telmisartan, valsartan, and EXP-3137, FI6828K, and RNH6270, and the like;

(9) α/β adrenergic blockers such as nipradilol, arotinolol and amosulalol, and the like;

(10) alpha 1 blockers, such as terazosin, urapidil, prazosin, tamsulosin, bunazosin, trimazosin, doxazosin, naftopidil, indoramin, WHP 164, and XEN010, and the like;

(11) alpha 2 agonists such as lofexidine, tiamenidine, moxonidine, rilmenidine and guanobenz, and the like;

(12) aldosterone inhibitors, and the like;

(13) angiopoietin-2-binding agents such as those disclosed in WO03/030833; and

(14) A-type and B-type natriuretic peptides, such as nesiritide (Natrecor), A-type natriuretic peptide (ANP), B-type natriuretic peptide (BNP), urodilatin (Ularitide) and the like.

### Dosages

The dosage of peptides of the invention depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

The peptides of the invention can be administered to a human at a dosage between 10 μg and 500 mg per day. In a further embodiment, the peptides may be administered between 100 μg and 100 mg per day. In yet a further embodiment, the peptides may be adminstered between 500 μg and 10 mg per day.

Continuous daily dosing with the peptides of the invention may not be required. A therapeutic regimen may require cycles, during which time a drug is not administered, or therapy may be provided on an as needed basis during periods of acute inflammation.

### Administration

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment.

In one aspect, the invention features the parenteral administration of a B isomer of a guanylin family peptide. In a further embodiment, the peptide may be administered intravenously, intramuscularly or subcutaneously. In yet a further embodiment, the peptide may be administered intravenously. Other routes of administration for the various embodiments include, but are not limited to, topical, transdermal, transcranial, nasal, and other forms of systemic administration (such as, inhalation, rectal, buccal, vaginal, intraperitoneal, intraarticular, ophthalmic, otic, or oral administration). As used herein, “systemic administration” refers to all non-dermal routes of administration, and specifically excludes topical and transdermal routes of administration.

### Experimental Results

Characterization and Stability of the huUgnA and B

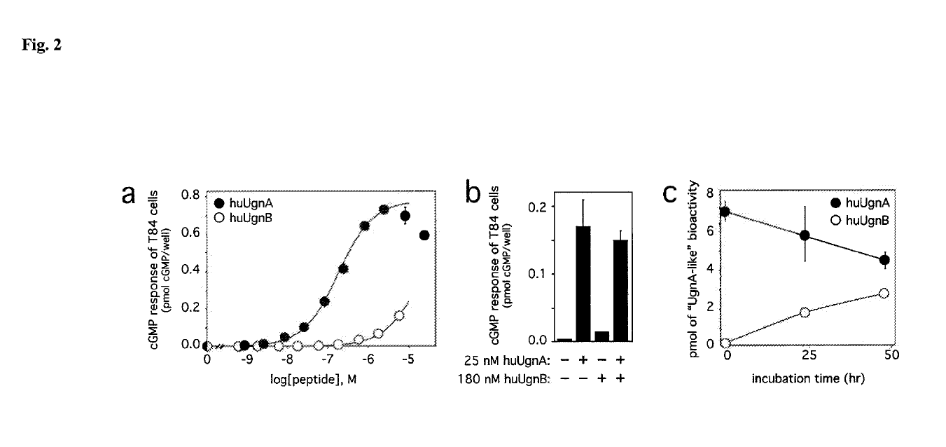

We used LC-MS to authenticate the initial identities and purities (>94%) of commercially-obtained huUgnA and B, and to verify at periodic intervals that conversion from one isoform to the other did not occur during storage at −80° C. We also confirmed that the A isoform could activate cyclic GMP synthesis in the GC-C-expressing T84 cell line, while the B isoform was more than 100-fold less potent (FIG. 2A). Furthermore, T84 cell responses evoked by co-application of huUgnA and B were not different from those evoked by huUgnA alone (FIG. 2B), indicating that the minimal responses to huUgnB reflect its intrinsically low agonist activity, rather than any antagonist properties at GC-C receptors.

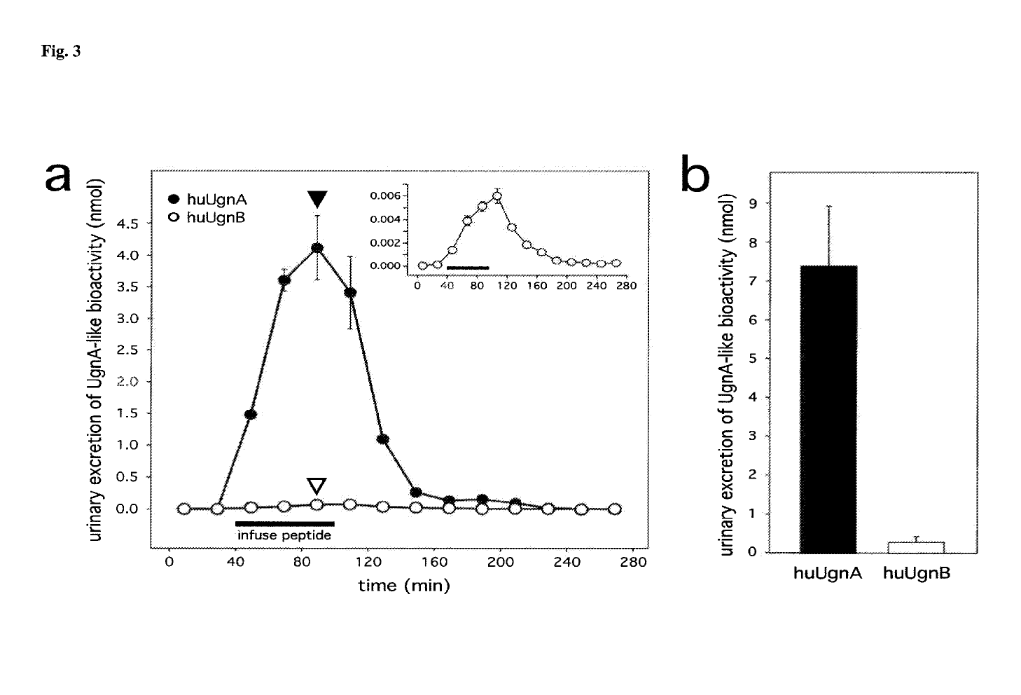

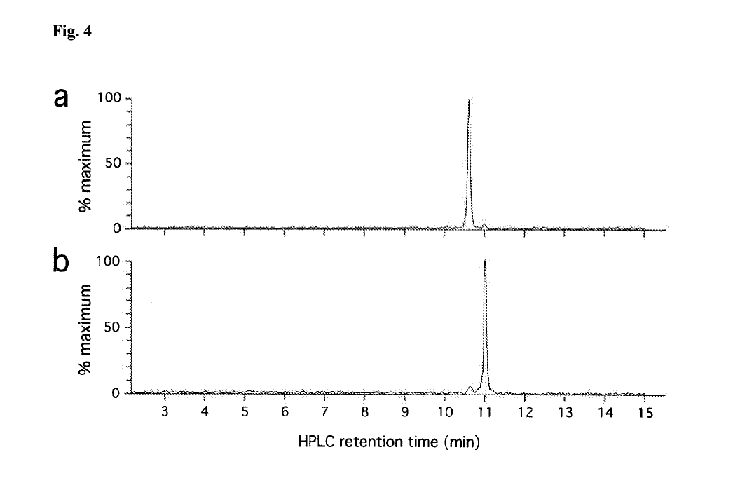

In contrast to the long-term stability of each individual isomer at −80° C., equilibration to a mixture of A and B forms occurs readily when either peptide is incubated at pH 4 and 50° C. (FIG. 2C). However, as has been well-documented in previous studies, significant isomerization requires many hours in vitro. To examine the possibility that some as-yet unidentified process might catalyze a more rapid interconversion of the isomers in vivo, we infused huUgnA or B intravenously into anesthetized rats and used both the T84 bioassay and LC-MS analysis to determine which form of the peptide was excreted in the urine. When huUgnA was infused, a significant amount of GC-C stimulating activity appeared in urine within 20 min of the start of the infusion, reached a peak by the end of the infusion, and returned to baseline within an hour after the infusion was terminated (FIG. 3A, black symbols). Comparable levels of urinary activity were recovered in 6 independent huUgnA infusion experiments (FIG. 3B, black bars). We then performed LC-MS analysis of urine obtained when peptide excretion was at its peak (marked by the black arrowhead in FIG. 3A), and observed a clearly discernable huUgnA signal along with a barely detectible huUgnB signal (FIG. 4A), indicating that essentially no isomerization had occurred within the animal.

In contrast, when animals were infused with huUgnB, very little GC-C-stimulating activity was recovered in the urine (FIG. 3A, white symbols, and FIG. 3B, white bars, n=6; the small response in FIG. 3A is resealed in the inset). The low levels of activity observed in the inset most likely represent weak responses to very high levels of huUgnB, as would be expected from the huUgnB concentration-response curve (FIG. 2A) and the well-known concentrating effect of the kidney (which generated a Ugn urine-to-plasma ratio of >500:1 in our studies). This interpretation was confirmed by the corresponding LC-MS analysis, which revealed a clearly discernable huUgnB signal along with a barely detectible huUgnA signal (FIG. 4B).

We then calibrated the LC-MS procedure with a control sample that contained approximately equal amounts of the A and B peptides (FIG. 1). The two isomers were detected with equal efficiency in the extracted ion chromatogram, arguing that the peak areas in FIGS. 4A and 4B provide an accurate assessment of the relative amounts of each isomer that were present in the urine. Taken together, these results indicate that infused huUgnA and huUgnB retain their identities and unique properties in vivo, and are sufficiently stable for use in acute animal studies.

Effects of huUgnB and huUgnA on Renal Sodium Excretion

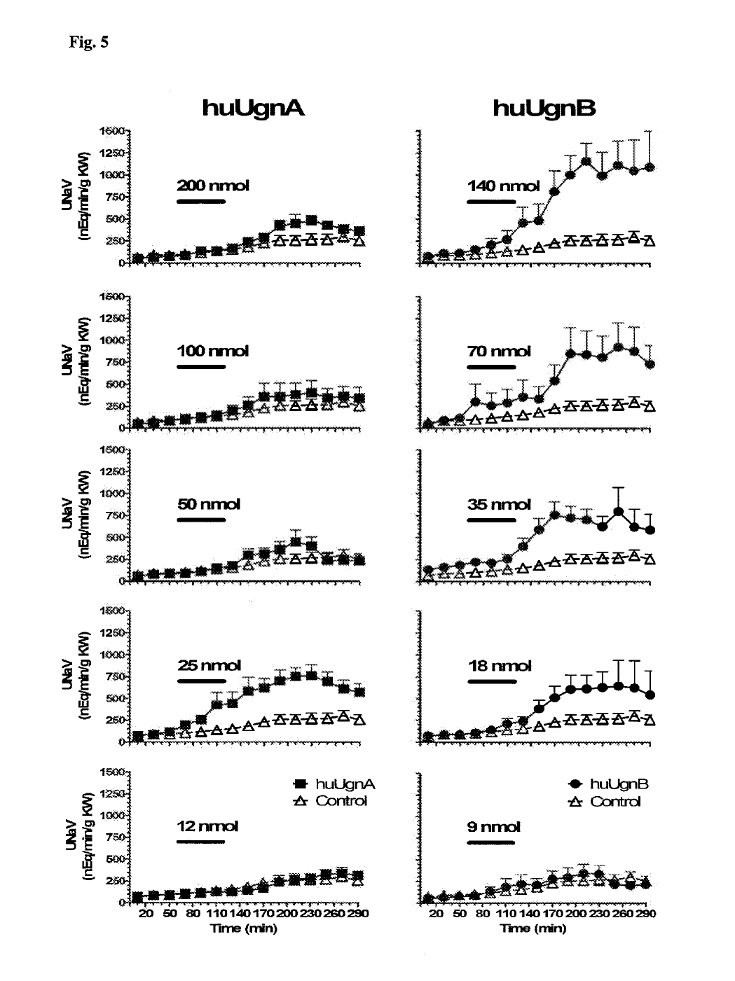

FIG. 5 illustrates the effects of the two huUgn isomers on urinary sodium excretion as a function of time, before, during, and after iv infusion into anesthetized animals. The effects of huUgnA are shown in the left panel, and those of huUgnB in the right. Prominent, dose-dependent natriuretic responses were observed, whereas the time control (saline infused) animals displayed relatively stable baseline levels of sodium excretion. The small increase in the control animals paralleled a small, but consistent, decline in blood pressure.

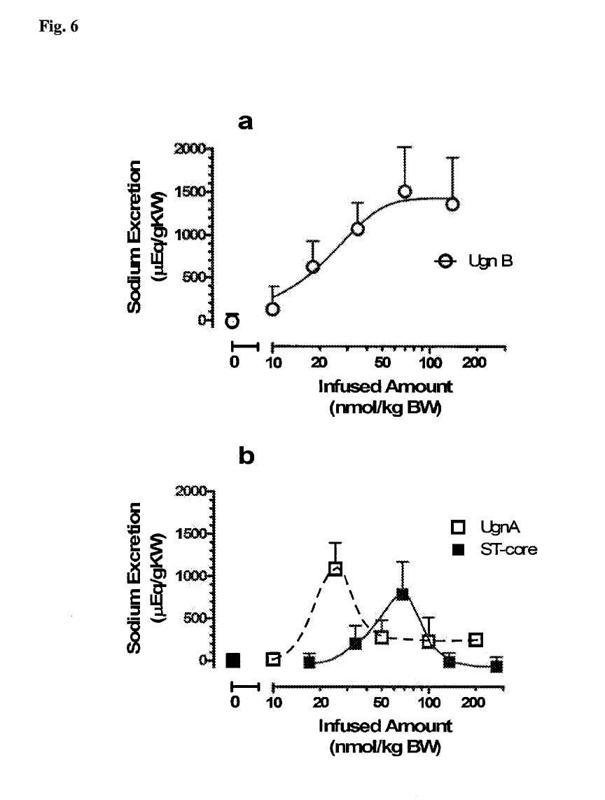

Interestingly, although huUgnA and B both produced a natriuresis, the dose-dependencies of the responses were quite different. The dose-response relationship for huUgnB (right panel) appeared to be conventional, with increasing doses causing greater natriuresis, while the response to huUgnA was unusual, in that the maximum response occurred at the relatively low dose of 25 nmol/kg and declined as doses increased. To examine this more quantitatively, we determined the cumulative sodium excretion evoked by each dose of each isomer (as described in the Methods section). When these net excretory values were analyzed, huUgnB produced significant natriuresis for all doses above 9 nmol, and the resulting curve was well fit by the log(agonist) vs response equation (see methods; p<0.02), with an ED₅₀ of about 20 nmol UgnB/kg BW (FIG. 6A). In contrast, huUgnA produced a bell-shaped dose-response relation with a single effective dose at 25 nmol/kg (FIG. 6B, open symbols). Lower or higher doses of huUgnA did not generate responses that could be distinguished statistically from control. This nearly complete loss of renal responsiveness to high concentrations of huUgnA is quite different from the modest drop observed when high doses of the peptide are applied to GC-C-expressing cells (FIG. 2B).

One striking similarity between the two peptides was the relatively long latency (˜50 min) and prolonged duration of the evoked response, with a prominent natriuresis still recognizable at the end of the 270 min observation period. The sluggish nature of these responses cannot be accounted for by an unexpectedly slow rate of peptide delivery to the kidney, since it is apparent by comparing FIG. 5 to FIG. 3A that the majority of the peptide-evoked natriuresis occurred long after the infused peptides had been cleared from the animal.

Mice also exhibit a natriuretic response to huUgnB.

The Natriuretic Dose-Response to huUgnA is Mimicked by ST-Core

The unconventional natriuretic dose-response relationship for huUgnA was unexpected, as no previous study has reported this kind of response to any peptide in the Gn/Ugn family. With this in mind, the infusion protocol was repeated with ST-core, a fragment of the heat stable enterotoxin produced by E. coli. This bacterial peptide is a structural and functional analog of UgnA, with an EC₅₀ for cyclic GMP stimulation in T84 cells that is about 10 fold lower than that of UgnA. If these relative potencies extend to the kidney, then the effects of ST-core should be similar, but slightly left-shifted, when compared to those of huUgnA. FIG. 6B compares the net sodium excretory responses evoked by ST-core (filled symbols) to those evoked by huUgnA (open symbols). The similarity between the overall response patterns is striking, but, surprisingly, ST-core appeared to be a less effective natriuretic factor than huUgnA: the maximal natriuresis elicited by ST-core was slightly less and the response was shifted to the right.

Responses to huUgnA and huUgnB are not Additive

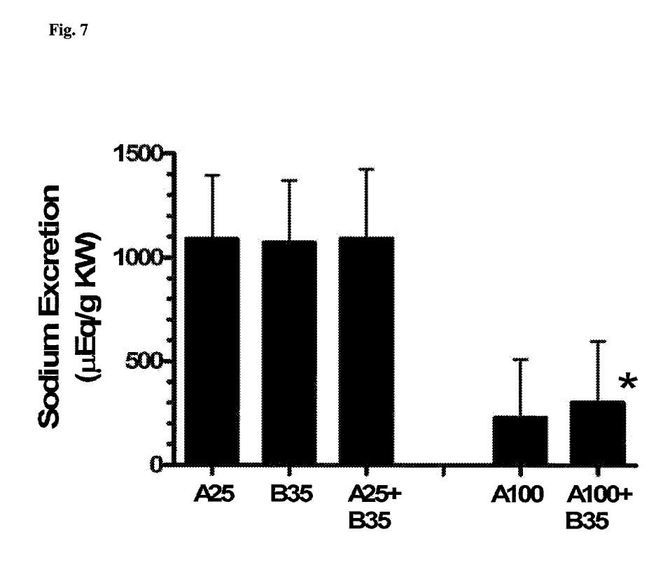

Given the distinctive nature of the response profiles obtained with the A and B isomers, it was of interest to determine how the two peptides would interact when applied simultaneously. To investigate this, animals were infused with mixed peptide solutions composed of huUgnB at 35 nmol/kg and huUgnA at either 25 or 100 nmol/kg. In this way, an effective dose of huUgnB was coupled with either an equipotent dose of huUgnA (25 nmol/kg), or a non-effective, supramaximal dose of huUgnA (100 nmol/kg). The results of these co-infusion protocols are shown in FIG. 7. Interestingly, combined maximal doses of huUgnA and B did not produce an additive response. Furthermore, the normal natriuretic response to 35 nmol huUgnB was almost completely suppressed when combined with the 100 nmol supramaximal dose of huUgnA, indicating a profound physiological interaction between the two peptides at high concentrations.

Effects of huUgnB and huUgnA on Other Physiological Parameters

Blood pressure, Renal Blood Flow and GFR. Blood pressure declined slightly over time in all animals, but did not fall below 100 mmHg in any group (Table 1). There were no significant differences between mean arterial pressures in control and experimental groups during the pre infusion, infusion, or post infusion time periods (Table 1).

Renal blood flow (RBF) was stable in control rats at 2.6+0.4 ml/min/g KW in the pre-infusion period and 2.5+0.5 ml/min/g KW in the post infusion period (n=4). RBF in huUgnA and B infused rats was not different from the control group during the pre infusion period, though small but significant increases in RBF occurred after 25 nmol of UgnA (2.7+0.3 to 3.2+0.1 ml/min/g KW, p<0.001, n=4) and after 35 nmol of huUgnB/kg (2.5+0.1 to 3.0+0.4 p<0.001 n=4). These changes corresponded to small, non-significant reductions in vascular resistance. GFR was stable in all groups (Table 1).

TABLE 1

| | | | Hour | | | | |
|---|---|---|---|---|---|---|---|
| | 1 | 2 | 3 | 1 | 2 | 3 | n |
| | Pre-infuse | Infusion | Post-infuse | Pre-infuse | Infuse | Post-infuse | ± |
| | UNaV nEq/min/g KW | | | FENa % of filtered | | | |
| Time Control | 84 ± 18 | 119 ± 17 | 188 ± 28 | 0.09 ± 0.02 | 0.12 ± 0.02 | 0.22 ± 0.03 | 10 |
| huUgnA 12 | 85 ± 20 | 118.1 ± 25 | 222 ± 33 | 0.08 ± 0.02 | 0.10 ± 0.02 | 0.17 ± 0.02 | 8 |
| huUgnA 25 | 93 ± 7 | 291.7 ± 77* | 664 ± 75*** | 0.09 ± 0.01 | 0.27 ± 0.08* | 0.68 ± 0.08*** | 8 |
| huUgnA 50 | 73 ± 10 | 117.5 ± 22 | 342 ± 63* | 0.06 ± 0.01 | 0.10 ± 0.01 | 0.28 ± 0.05 | 14 |
| huUgnA 100 | 66 ± 9 | 130.8 ± 32 | 328 ± 80 | 0.06 ± 0.01 | 0.12 ± 0.03 | 0.27 ± 0.06 | 5 |
| huUgnA 200 | 67 ± 1 | 120.6 ± 18 | 342 ± 51 | 0.06 ± 0.01 | 0.11 ± 0.02 | 0.29 ± 0.03 | 3 |
| huUgnB 9 | 67 ± 17 | 134.6 ± 53 | 195 ± 37 | 0.05 ± 0.01 | 0.09 ± 0.02 | 0.21 ± 0.04 | 4 |
| huUgnB 18 | 83 ± 19 | 155.2 ± 34 | 538 ± 81*** | 0.07 ± 0.01 | 0.12 ± 0.03 | 0.42 ± 0.08 | 10 |
| huUgnB 35 | 132 ± 40 | 229.4 ± 47 | 729 ± 73*** | 0.12 ± 0.02 | 0.27 ± 0.07 | 0.62 ± 0.10*** | 10 |
| huUgnB 70 | 87 ± 31 | 285.7 ± 165 | 696 ± 151*** | 0.08 ± 0.03 | 0.24 ± 0.14 | 0.52 ± 0.10* | 8 |
| huUgnB 140 | 103 ± 8 | 212.1 ± 67 | 906 ± 170*** | 0.08 ± 0.01 | 0.18 ± 0.05 | 0.65 ± 0.11** | 3 |
| | UKV nEq/min/g KW | | | FEK % of filtered | | | |
| Time Control | 640 ± 69 | 1096 ± 64 | 1088 ± 66 | 22.7 ± 2.3 | 32.9 ± 2.4 | 36.6 ± 2.3 | 10 |
| huUgnA 12 | 709 ± 74 | 1097 ± 54 | 1299 ± 51 | 18.8 ± 2.3 | 29.8 ± 3.6 | 34.9 ± 2.7 | 8 |
| huUgnA 25 | 780 ± 69 | 1292 ± 80 | 1309 ± 68 | 25.6 ± 2.4 | 40.7 ± 3.5 | 46.5 ± 3.8 | 8 |
| huUgnA 50 | 722 ± 52 | 988 ± 66 | 1180 ± 57 | 20.8 ± 2.1 | 32.0 ± 2.7 | 36.3 ± 3.6 | 14 |
| huUgnA 100 | 602 ± 51 | 878 ± 60 | 970 ± 72 | 19.3 ± 2.5 | 28.0 ± 3.5 | 29.9 ± 2.9 | 5 |
| huUgnA 200 | 860 ± 118 | 1203 ± 161 | 1380 ± 73 | 27.7 ± 5.9 | 40.8 ± 7.7 | 57.5 ± 6.7*** | 3 |
| huUgnB 9 | 655 ± 79 | 840 ± 83 | 943 ± 82 | 23.4 ± 6.0 | 30.6 ± 6.1 | 34.8 ± 3.1 | 4 |
| huUgnB 18 | 592 ± 71 | 860 ± 72 | 1014 ± 79 | 20.0 ± 2.3 | 29.6 ± 3.2 | 39.2 ± 2.6 | 10 |
| huUgnB 35 | 729 ± 79 | 837 ± 84 | 998 ± 85 | 25.7 ± 2.4 | 32.6 ± 3.7 | 38.9 ± 3.0 | 10 |
| huUgnB 70 | 791 ± 265 | 1297 ± 209 | 1491 ± 183* | 20.4 ± 5.5 | 37.7 ± 9.8 | 47.1 ± 5.6 | 8 |
| huUgnB 140 | 770 ± 78 | 1086 ± 124 | 1396 ± 89 | 28.3 ± 0.9 | 49.2 ± 3.6 | 58.4 ± 3.5** | 3 |

Mean values ± SEM of sodium and potassium excretion in the 60 min before peptide infusion (Pre-infuse), during 60 min of huUgnA or huUgnB infusion (Infusion) and 90 minutes after peptide infusion (Post-infuse). The doses of peptide infused are shown in nmol. Experimental values in each column were tested against corresponding values in the time control group by ANOVA and the Bonferroni method for selected multiple comparisons.
*= p < 0.05,
**= p < 0.01,
***= p < 0.005

Diuresis. A slight increase in urine flow occurred in all groups during the first hour of the protocol (Table 2). In control rats, for example, urine flow increased slightly from 2.1±0.2 in the pre infusion period to 2.6±0.2 μl/min/g KW in the infusion period (Table 2). However, all experimental groups showed this same pattern, with no statistically significant differences among them. Similarly, urine flow in all peptide infused rats was not different from the control group during peptide infusions at all doses tested. However, urine flow was significantly higher than control in the post infusion period at several doses of huUgnA and B, most notably at the high ends of the dose-response curves (Table 2).

TABLE 2

| | | | | | | | GFR μl/min/g | | | |
|---|---|---|---|---|---|---|---|---|---|---|
| | BP mmHg | | | V μl/min/g KW | | | KW | | | |
| | 1 | | 3 | 1 | | 3 | 1 | | 3 | |
| | Pre- | 2 | Post- | Pre- | 2 | Post- | Pre- | 2 | Post- | |
| | infuse | Infusion | infuse | infuse | Infusion | infuse | infuse | Infusion | infuse | n |
| Time Control | 123 ± 3 | 114 ± 3 | 109 ± 5 | 2.12 ± 0.17 | 2.60 ± 0.18 | 2.77 ± 0.23 | 814 ± 77 | 882 ± 71 | 901 ± 69 | 10 |
| huUgnA 12 | 121 ± 4 | 110 ± 4 | 104 ± 3 | 2.07 ± 0.23 | 2.34 ± 0.14 | 2.73 ± 0.14 | 895 ± 51 | 869 ± 64 | 923 ± 66 | 8 |
| huUgnA 25 | 119 ± 3 | 111 ± 4 | 105 ± 4 | 2.13 ± 0.17 | 2.98 ± 0.28 | 3.53 ± 0.25* | 820 ± 40 | 886 ± 66 | 823 ± 49 | 8 |
| huUgnA 50 | 117 ± 3 | 106 ± 3 | 101 ± 3 | 2.01 ± 0.13 | 2.32 ± 0.16 | 3.07 ± 0.20 | 858 ± 52 | 817 ± 53 | 869 ± 49 | 14 |
| huUgnA 100 | 121 ± 4 | 115 ± 3 | 109 ± 4 | 1.76 ± 0.22 | 2.48 ± 0.26 | 2.67 ± 0.23 | 807 ± 62 | 858 ± 55 | 906 ± 38 | 5 |
| huUgnA 200 | 121 ± 7 | 113 ± 5 | 112 ± 4 | 2.14 ± 0.39 | 2.84 ± 0.40 | 4.12 ± 0.35** | 857 ± 43 | 827 ± 61 | 849 ± 24 | 3 |
| huUgnB 9 | 115 ± 5 | 108 ± 6 | 107 ± 5 | 1.84 ± 0.28 | 2.22 ± 0.19 | 2.55 ± 0.18 | 921 ± 57 | 918 ± 66 | 947 ± 60 | 4 |
| huUgnB 18 | 124 ± 2 | 117 ± 3 | 113 ± 3 | 1.79 ± 0.14 | 2.40 ± 0.15 | 3.11 ± 0.32 | 890 ± 55 | 936 ± 76 | 877 ± 80 | 10 |
| huUgnB 35 | 118 ± 3 | 108 ± 4 | 107 ± 3 | 2.38 ± 0.25 | 2.53 ± 0.16 | 3.84 ± 0.27*** | 866 ± 64 | 830 ± 48 | 870 ± 53 | 10 |
| huUgnB 70 | 119 ± 3 | 107 ± 5 | 107 ± 4 | 2.04 ± 0.20 | 3.11 ± 0.71 | 3.92 ± 0.5* | 883 ± 95 | 867 ± 86 | 876 ± 26 | 8 |
| huUgnB 140 | 123 ± 5 | 119 ± 5 | 117 ± 5 | 2.24 ± 0.14 | 2.71 ± 0.40 | 4.50 ± 0.80** | 858 ± 86 | 788 ± 67 | 924 ± 46 | 3 |

Mean values ± SEM of Mean arterial blood pressure (BP, urine flow rate per min (V), and glomerular filtration rate per min (GFR) during the 60 min before peptide infusion (Pre-infuse), during 60 min of huUgnA or huUgnB infusion (Infusion) and 90 minutes after peptide infusion (Post-infuse). The doses of peptide infused are shown in nmol. The number of animals in each group is shown in parentheses. Experimental values in each column were tested by ANOVA against corresponding values in the time control group using the Bonferroni method for selected multiple comparisons.
*= p < 0.05,
**= p < 0.01,
***= p < 0.005

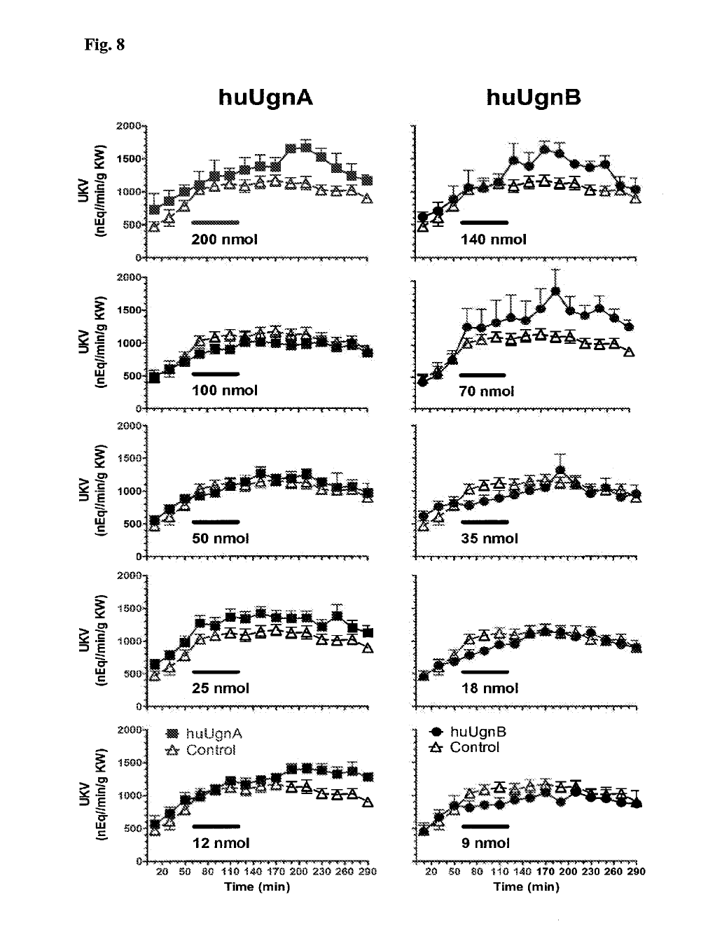

Kaliuresis. Kaliuretic effects of huUgnA and B are summarized in Table 1 and FIG. 8, and are much less pronounced than the natriuretic effects of the peptides. Indeed, no statistically significant increases in UKV were observed during or after huUgnA infusions at any dose tested, although the highest dose of huUgnA tended to evoke a response (p>0.05). For huUgnB, only the 70 nmol/kg dose produced a consistent increase in UKV, which occurred in the post infusion period. FEK was increased over control levels in the post infusion period, but only after of the highest doses of huUgnA and B. This right-shifted dose-dependency for kaliuresis (relative to the natriuretic effects of the peptides) raises the possibility that the diuretic and kaliuretic effects are mediated by a receptor distinct from the receptor that elicits the natriuretic response.

### Experimental Methods

Experiments were performed on 139 male Sprague Dawley rats obtained from Charles River Breeding Laboratories, Raleigh N.C. Animals were maintained on a 12 hour light/dark cycle with free access to water and standard rat chow. All experimental procedures were approved by the Institutional Animal Care and Use Committee at the University of North Carolina at Chapel Hill, and were carried out in accordance with the NIH Guide for the Care and Use of Laboratory Animals.

### Rat Preparation

Rats were not fasted prior to clearance experiments and ranged in weight from 250-340 g at the time of study. Anesthesia was induced with an intraperitoneal injection of pentobarbital sodium (55 mg/kg BW) and maintained at a surgical plane by intermittent intravenous supplementation. Core temperature was maintained at 37° C. by a servo-controlled, heated operating table. Animals breathed spontaneously through a PE240 cannula inserted into the trachea. A jugular vein was catheterized with PE50 cannula tubing for intravenous infusion of isotonic saline containing FITC-labeled inulin (0.4% W/V, Sigma, St Louis Mo.) and two PE10 cannulae, one for supplemental anesthetic and the other for peptide infusions (described below in the experimental protocol section). The ureters were exposed through a midline ventral incision and cannulated with PE10 tubing about one cm from each kidney. Urine was collected over 20 min periods and the volume estimated by weight. A femoral artery was cannulated with PE50 tubing for continuous measurement of arterial blood pressure and intermittent blood samples (50 μl), taken at the mid point of each urine collection.

All rats received a continuous intravenous infusion (10 μl/min/100 g BW) of isotonic saline containing FITC-labeled inulin for measurement of inulin clearance. Uncongugated FITC label and small inulin polymers were removed from the inulin solution by overnight dialysis against isotonic saline (SpectraPor 6 dialysis membrane, 1 kDa cutoff-limit, Spectrum Laboratories, Rancho Domingo, Calif.). Undissolved particulates were removed from the solution by filtration through a 0.2 μM filter before each experiment (Steriflip, Millipore Inc. Billerica, Mass.). At least one hr was allowed for equilibration before the start of urine collections, at which point plasma inulin concentration had stabilized at an average value of 30±3 μg/ml. This procedure resulted in a hydropenic state in which renal function is consistent with the homeostatic response to salt and water restriction.

Arterial blood pressure was measured with a pressure transducer connected to a cardiovascular analyzer (Model 50110, Stoelting Instruments Wood Dale, Ill.). Left renal blood flow was measured in some rats with a flow probe positioned on the left renal artery and connected to a blood flow monitor (model 1 PRB probe and model T420 monitor, Transonic Systems Inc. Ithaca N.Y.). Arterial pressure, heart rate, and renal blood flow were digitized with an A/D converter (Keithley instruments, Cleveland, Ohio Model KUSB 3100) for display and storage on a Windows-based personal computer using open layers data acquisition software (Dtx_Ez, Data Translation Inc, Marlboro, Mass.). Sodium and potassium concentrations in plasma and urine were measured by flame photometry (Model 943, Instrumentation Laboratory Co., Lexington Mass.). Glomerular filtration rate (GFR) was measured as the renal clearance rate of FITC-labeled inulin

### Experimental Protocols

After post-surgical equilibration (−60 min) and three 20 min control clearance periods, huUgnA or B was infused into the jugular vein at 10 μl/min over a 60 min period, followed by a return to isotonic saline for the remainder of the experiment. The infused doses of huUgnA were 12, 25, 50, 100, or 200 nmol/kg BW in a total of 38 rats; huUgnB doses were 9, 18, 35, 70, and 140 nmol/kg BW in 35 rats. A UgnA mimetic, E. coli STh (ST-core), was infused into a third group of 32 rats at doses of 17, 32, 66, 133, and 266 nmol/kg. Renal excretory responses to these infusions were slow to develop and long lasting, so clearance periods were continued for 2-3 hr after the termination of the peptide infusion. This protracted time course limited protocols to one dose of one peptide in each rat. A control group of 10 rats received isotonic saline at 10 μl/min in place of the peptide infusion, but was otherwise treated in the same way as the experimental groups.

The effects of combined infusions of huUgnA and B were investigated in two groups of rats that received 35 nmol/kg of huUgnB together with either 25 nmol/kg (n=5) or 100 nmol/kg of huUgnA (n=5). The experimental protocol was otherwise the same as that for the individual peptide infusions.

### Peptides Used

huUgnA and huUgnB were obtained from a commercial supplier (Bachem Americas Inc. Torrance, Calif.). ST-core was provided by Ironwood Pharmaceuticals Inc (Cambridge, Mass.). These peptides were dissolved at a concentration of 1 μg/μl in sterile saline containing 0.1% BSA. This amounted to 500 pmol/ml for huUgnA, 350 pmol/ml for huUgnB, and 670 pmol/ml for ST-core. Aliquots of these solutions were stored at −80° C., then thawed and diluted into 0.6 ml of isotonic saline to provide the desired concentration immediately prior to infusion into the animal.

### Inulin Assay

This assay was based on the method described by Lorenz and Gruenstein. Urine samples were diluted with hepes-buffered saline (3, 5, 10, or 20 fold, based on urine flow) to neutralize the pH and bring the intensity of FITC emissions into detector range. Small volumes (3-5 μl) of diluted urine and undiluted plasma samples were drawn into constant bore capillary tubes (10 μl Microcap tubes, Drummond Scientific Company, Broomall Pa.) and sealed at each end with water-saturated mineral oil. FITC emission intensity was measured by imaging each sample at 10× magnification with an epifluorescence microscope (Axiovert S100TV, Carl Zeiss Inc. Thornwood, N.Y.) fitted with a CCD camera (Orca II, Hamamatsu Inc. Bridgewater, N.J.) controlled by Metamorph imaging software (Molecular Devices, Sunnyvale, Calif.). Fluorescence intensities were measured as mean pixel intensity obtained from a consistent central region of each sample during a 25 or 500 msec exposure for urine and plasma, respectively. Background intensities were measured in the same way using bladder urine and tail vein plasma collected from the same rat before the beginning of inulin infusion. Inulin concentrations in plasma and urine samples were calculated from a standard curve prepared from the infusion solution used in each experiment. A highly significant linear relationship between fluorescence intensity and inulin concentration was obtained in each assay (r²=0.993, p<0.001 for 16 representative assays).

### Ugn Assays

HPLC analysis and mass spectroscopy of Ugn isomers. The integrity and purity of huUgnA and B isomer preparations were confirmed by liquid chromatography/mass spectroscopy (LC-MS) analysis. The two Ugn isomers have different retention times on a reverse phase HPLC column (FIG. 1, dashed trace; huUgnA elutes ˜0.35 minutes before huUgnB). Separations were performed on a Hypersil Gold AQ 2.1×50 mm column from Thermo Fisher Scientific Inc (Waltham, Mass.) equilibrated in 98% buffer A (0.1% formic acid), 2% buffer B (85:10:5 Acetonitrile/Isopropyl Alcohol/5 mM NH₄OAc pH 5.8) with a flow rate of 0.4 ml/min on an Acquity HPLC system. After a 2.5 minute wash with the same buffers, peptides were eluted with a linear gradient of 2% buffer B to 80% buffer B over 25 minutes and held for one minute before an increase to 90% B over 2 minutes to wash the column, followed by a return to 2% buffer B over 3 minutes.

Peptide masses were determined using a Micromass Q-T of II instrument equipped with an electrospray ionization (ESI) source operating in positive ion mode. The instrument was programmed to scan in the mass range of m/z 100 to 1800. Molecular weight predictions and data analysis were carried out with MassLynx version 4.0 software. 200 of urine were injected directly without any sample preparation using an Acquity HPLC system connected in line with the Q-T of II. In control experiments, comparable recoveries were obtained for huUgnA and huUgnB standards (FIG. 1, compare peak areas in the dashed trace (the extracted chromatogram) to those in the solid trace (the UV absorbance)), thus validating the use of the LC-MS technique for quantitative comparisons of peptide recoveries in urine after iv infusion.

Bioassay of huUgnA-like activity. The concentration of huUgnA-like bioactivity in infused peptide solutions and in urine collected during experiments was measured using a T84 cell-based bioassay, as described previously. T84 cells are a colon carcinoma cell line that increases cyclic GMP production in response to GC-C receptor agonists such as Ugn, Gn and ST core peptide. T84 cells were grown to near confluence in 12 well culture clusters, and then incubated with unknowns or standard concentrations of huUgnA. Standards or urine samples were diluted into bioassay medium (1 mM 3-isobutyl-1-methylxanthine, 0.03 mM phenol red, 137 mM NaCl, 5.4 mM KCl, 0.25 mM Na2HPO4, 0.44 mM KH2PO4, 1.3 mM CaCl2, 1.0 mM MgSO4, 4.2 mM NaHCO3, 10 mM HEPES buffered at pH 7.0) and the pH was adjusted to 7.0, if required, using the phenol red indicator dye as a guide. This pH adjustment was necessary, as it is well-established that the response of T84 cells to UGn is highly pH dependent. The broad spectrum phosphodiesterase inhibitor (isobutyl methylxanthine) was included to prevent degradation of cyclic GMP. After 30 min, the cells were lysed and cyclic GMP levels measured by radioimmunoassay (Biomedical Technologies Inc Stoughton Mass.). Standards and unknowns were assayed in triplicate. The increased cyclic GMP levels induced by unknown samples were converted to huUgnA concentrations by interpolation into the standard curve that was generated from the huUgnA standard solutions, and are reported as μmol Ugn-like activity per well (mean±sem). The responses evoked by the standards were fit using the log(agonist) vs response equation given below.

### Data Analysis

Sodium and potassium excretion rates are expressed as absolute values (UNaV and UKV) or factored by filtered load to provide fractional excretion (FENa and FEK). Glomerular filtration rate (GFR) was equated with inulin clearance. The net natriuretic response to peptide infusions was assessed by summing total Na excretion during infusion and post-infusion collection periods in each rat after subtraction of corresponding mean values obtained from the control group. The cumulative net excretion obtained in this way provides a measure of the natriuretic response to each dose of peptide infused. Results from both kidneys were averaged for each rat before calculating group means±sem.

Group comparisons were made with one way analysis of variance (ANOVA) using peptide doses as column variables. Columns were further divided into pre-infusion, infusion, and post infusion subgroups to facilitate post hoc testing with Bonferroni's method for selected multiple comparisons. Row variables were individual measurements from each clearance period for each rat. Responses were tested for statistical significance by comparing control values during the pre-infusion, infusion and post infusion collection periods with corresponding values obtained from peptide-infused rats. Each experimental group included 5 different doses of huUgnA, huUgnB, or ST-core. Thus, fifteen comparisons were required for each measured variable in each group.

All graphing and statistical testing were performed with the Prism 5.01 graphing and analysis program (Graphpad Software, San Diego, Calif.). Dose-response curves for huUgnA- and ST-core-evoked net sodium excretion were fit using the spline algorithm. Dose-response curves for huUgnB-evoked net sodium excretion and for T84 cell bioassay responses were fit using the log(agonist) vs response equation:

(response at each dose)=(control response)+((maximal response−control response)/[1+10̂(log(ED₅₀)−log(dose_{Ugn}))]).

### Other Embodiments

Various modifications and variations of the described methods and compositions described herein will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method for treating a disorder characterized by fluid retention in a subject, said method comprising administering an effective amount of composition comprising a peptide, wherein said peptide is the B isomer of a guanylin family peptide, wherein said B isomer peptide is present in said composition at a non-naturally occurring ratio with the A form of said peptide.

2. The method according to claim 1, wherein said guanylin family peptide is a uroguanylin (Ugn) peptide or a guanylin (Gn) peptide.

3. The method according to claim 2, wherein said method comprises administering an effective amount of composition comprising the B isomer of Ugn (UgnB), wherein said UgnB is present in said composition at a non-naturally occurring ratio with the A form of Ugn (UgnA).

4-6. (canceled)

7. The method of claim 3, wherein said UgnB is modified to decrease the rate of conversion to UgnA.

8. The method according to claim 2, wherein said method comprises administering an effective amount of composition comprising the B isomer of Gn (GnB), wherein said GnB is present in said composition at a non-naturally occurring ratio with the A form of Gn (GnA).

9-12. (canceled)

13. The method according to claim 1, wherein said peptide has an amino acid sequence of:
(SEQ ID NO: 2)
Xaa₁-Xaa₂-Xaa₃-Cys-Glu-Xaa₄-Cys-Xaa₅-Xaa₆-Xaa₇-
Ala-Cys-Xaa₈-Xaa₉-Cys-Xaa₁₀-Xaa₁₁-Xaa₁₂;
wherein
Xaa₁ is Gly, Asn, Pro, Gln, Ser, Thr, Ala, Val, Leu, Ile, Met, Phe, Trp, Tyr or is absent;
Xaa₂ is Asp, Glu, Gly, His, Asn, Ser, Gln, Thr or is absent;
Xaa₃ is Thr, Glu, Asp, or Ser;
Xaa₄ is Be or Leu;
Xaa₅ is Val, Be, Ala, or Leu;
Xaa₆ is Asn, Tyr, Phe, or Gln;
Xaa₇ is Val, Be, Ala, Leu or Pro;
Xaa₈ is Ala, Ser or Thr;
Xaa₉ is Gly or Ala;
Xaa₁₀ is Leu, Be, Phe, Trp or Tyr;
Xaa₁₁ is Arg, Lys, Ala, Leu, Val, Ile, Ser, Thr, Met, Phe, Trp, Tyr, Asp, Glu, Gln, Asn or is absent; and
Xaa₁₂ is Arg, Lys, Ala, Leu, Val, Ile, Ser, Thr, Met, Phe, Trp, Tyr, Asp, Glu, Gln, Asn or is absent.

14-24. (canceled)

25. The method of claim 1, wherein the fluid retention disorder is selected from the group consisting of kidney disease, heart disease, liver disease, and hypertension.

26. The method according to claim 25, wherein said fluid retention disorder is selected from heart failure, hypertension, salt dependent forms of high blood pressure, hepatic edema, liver cirrhosis, acute renal failure, renal insufficiency, nephrotic edema, glomerulonephritis, pyelonephritis, kidney failure, chronic renal failure, nephritis, nephrosis, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic kidney disease, medullary cystic disease, medullary sponge kidney, hereditary nephritis, and nail-patella syndrome.

27. The method according to claim 26, wherein said fluid retention disorder is heart failure.

28. (canceled)

29. The method according to claim 26, wherein said fluid retention disorder is polycystic kidney disease.

30. (canceled)

31. The method according to claim 1, wherein said method increases natriuresis and/or diuresis.

32. The method according to claim 1, wherein the effective amount of the peptide is administered in combination with one or more additional drugs that affect salt balance, fluid balance, or both salt and fluid balance.

33. The method of claim 32, wherein said one or more other drugs comprises a diuretic.

34-35. (canceled)

36. A composition comprising a peptide, wherein said peptide is the B isomer of a guanylin family peptide, wherein said B isomer peptide is present in said composition at a non-naturally occurring ratio with the A form of said peptide; with the proviso that said peptide is not NDDCELCVNVACTGCL, PGTCEICAYAACTGCL, NDDCELCVNVACTGCLKK, ADDCELCVNVACTGCL, NDDCELCANVACTGCL, NDDCELCVNAACTGCL, NDDCELCVNVACAGCL, NDDCELCVNVACTACL, NDDCELCAYAACTGCL, or NDDCELCVNPACTGCL.

37. The composition according to claim 36, wherein said guanylin family peptide is an uroguanylin (Ugn) peptide or a guanylin (Gn) peptide.

38. The composition according to claim 37, wherein said composition comprises the B isomer of Ugn (UgnB), wherein said UgnB is present in said composition at a non-naturally occurring ratio with the A form of Ugn (UgnA).

39-41. (canceled)

42. The composition of claim 38, wherein said UgnB is modified to decrease the rate of conversion to UgnA.

43. The composition according to claim 42, wherein said composition comprises the B isomer of Gn (GnB), wherein said GnB is present in said composition at a non-naturally occurring ratio with the A form of Gn (GnA).

44. The composition of claim 43, wherein said composition comprises a ratio of GnB to GnA of greater than 9:1.

45-47. (canceled)

48. The composition according to claim 36, wherein said peptide has an amino acid sequence of:
(SEQ ID NO: 2)
Xaa₁-Xaa₂-Xaa₃-Cys-Glu-Xaa₄-Cys-Xaa₅-Xaa₆-Xaa₇-
Ala-Cys-Xaa₈-Xaa₉-Cys-Xaa₁₀-Xaa₁₁-Xaa₁₂;
wherein
Xaa₁ is Gly, Asn, Pro, Gln, Ser, Thr, Ala, Val, Leu, Ile, Met, Phe, Trp, Tyr or is absent;
Xaa₂ is Asp, Glu, Gly, His, Asn, Ser, Gln, Thr or is absent;
Xaa₃ is Thr, Glu, Asp, or Ser;
Xaa₄ is Be or Leu;
Xaa₅ is Val, Be, Ala, or Leu;
Xaa₆ is Asn, Tyr, Phe, or Gln;
Xaa₇ is Val, Be, Ala, Leu or Pro;
Xaa₈ is Ala, Ser or Thr;
Xaa₉ is Gly or Ala;
Xaa₁₀ is Leu, Be, Phe, Trp or Tyr;
Xaa₁₁ is Arg, Lys, Ala, Leu, Val, Ile, Ser, Thr, Met, Phe, Trp, Tyr, Asp, Glu, Gln, Asn or is absent; and
Xaa₁₂ is Arg, Lys, Ala, Leu, Val, Ile, Ser, Thr, Met, Phe, Trp, Tyr, Asp, Glu, Gln, Asn or is absent.

49-59. (canceled)
